(19) United States
(12) Patent Application Publication
(10) Pub. No.: US 2023/0368868 A1
(43) Pub. Date: Nov. 16, 2023

(54) ENTITY SELECTION METRICS

(71) Applicant: BenevolentAI Technology Limited, London (GB)

(72) Inventors: Gabi GRIFFIN, Brooklyn, NY (US); Nicholas LITOMBE, Brooklyn, NY (US); Daniel Paul SMITH, London (GB); Alexander DEGIORGIO, London (GB)

(21) Appl. No.: 18/359,093

(22) Filed: Jul. 26, 2023

Related U.S. Application Data
(63) Continuation of application No. PCT/GB2022/050130, filed on Jan. 18, 2022.
(60) Provisional application No. 63/141,696, filed on Jan. 26, 2021.

(51) Int. Cl. G16B 40/00; G16B 50/30; G16B 15/30

(52) U.S. Cl. CPC G16B 40/00; G16B 50/30; G16B 15/30

(57) ABSTRACT

Embodiments of present disclosure provide a system, apparatus and method(s) for generating a set of metrics for evaluating entities used with a predictive machine learning model, the method comprising: selecting one or more sets of entities from a data sources for generating a plurality of predictions aggregated from said one or more sets of entities using one or more pre-trained predictive models; selecting a subset of predictions from the plurality of predictions based on said one or more sets of entities in relation to the data source; extracting metadata from the data source associated with the subset of predictions, where the metadata comprises entity metadata and predicted metadata; generating the set of metrics based on the metadata extracted and the subset of predictions; and outputting the set of metrics for evaluation.

### CROSS-REFERENCE TO RELATED APPLICATIONS

The present application is a bypass continuation of International Application No. PCT/GB2022/050130, filed Jan. 18, 2022, which in turn claims the priority benefit of U.S. Application No. 63/141,969, filed Jan. 26, 2021. Each of these applications is incorporated herein by reference in its entirety for all purposes.

### FIELD OF INVENTION

The present application relates to a system, apparatus and method(s) for generating a set of metrics for evaluating and presenting entities, where the set of metrics is used with a predictive machine learning model.

### BACKGROUND

Knowledge graphs (KGs) are stores of information in the form of entities and the relationships between those entities. They are a type of data structure used to model an area of knowledge and help researchers and experts study the connections between entities of such an area. Predictive machine learning models are commonly implemented using KGs to generate new (inferred) connections between entities based on existing data. For example, in a KG covering biomedical knowledge, a disease and a gene may each be represented by an entity, while the relationship between the disease and gene is represented by the relation between the two entities. Expanding on this, predictive models may use another disease's similarities to the first disease to predict a certain ‘relation’ between the gene entity and the second disease entity. The ‘relation’ represents a potential interaction between the gene and the disease in the body, the knowledge of which—for instance—may help treat the disease. These relations are only predictions of physical scenarios so are often associated with a confidence score indicating their likelihood of manifesting in real-life.

Researchers may want to direct the predictive models to study and compute any relation in a specific area of the KG by pre-selecting entities to be investigated. For example, researchers may wish to explore a particular disease and the surrounding mechanisms by selecting a disease entity on a biomedical KG. The entity selected may yield, provided the number of predictive models available, yet still too many similar or related entities making the quality assessment of the results difficult without further manual analysis. Thus, streamlining the optimisation or effective selection of predictive machine learning models is imperative.

Present methods for optimising or selecting predictive machine learning models fall into one of three general categories: 1) evaluation of predictive model's efficacy; 2) a comparison of different predictive models or different configurations of a single model; and 3) assessment of the quality of the data stored in the KG that is to be used in a model.

However, none of the methods from the above categories effectively assess and compare the suitability of the initial entities that were inputted, but rather evaluate only the model. In other words, none of these methods allows a user to efficiently compare the impact that using different input entities has on a given model.

Accordingly, it is desired to develop a method, system, medium and/or apparatus, that can address at least the above issues and effectively assess and compare the suitability of the initial entities or which entities produce the most useful results given the model.

It is further understood that the embodiments described below are not limited to implementations which solve any or all of the disadvantages of the known approaches described above.

### SUMMARY

This Summary is provided to introduce a selection of concepts in a simplified form that are further described below in the Detailed Description. This Summary is not intended to identify key features or essential features of the claimed subject matter, nor is it intended to be used to determine the scope of the claimed subject matter; variants and alternative features which facilitate the working of the invention and/or serve to achieve a substantially similar technical effect should be considered as falling into the scope of the invention disclosed herein.

The present disclosure provides a user with comparison metrics for entity evaluation and an interface thereof. The metrics are constructed based on data from the knowledge graph and results predicted by machine learning or predictive models. The metrics adapt to the predictions from the models in an interactive manner. The user may select from the knowledge graph entities to be assessed using the metrics and the models. Based on the metrics, top entities may be identified and analysed further by the user. The metrics interface allows the user to interface the predictions with improved efficiency.

In a first aspect, the present disclosure provides computer-implemented method of generating a set of metrics for evaluating entities used with a predictive machine learning model, the method comprising: selecting one or more sets of entities from a data source; generating a plurality of predictions aggregated from said one or more sets of entities using one or more pre-trained predictive models; selecting a subset of predictions from the plurality of predictions based on said one or more sets of entities in relation to the data source; extracting metadata from the data source associated with the subset of predictions, wherein the metadata comprises entity metadata and predicted metadata; generating the set of metrics based on the metadata extracted and the subset of predictions; and outputting the set of metrics for evaluation.

In a second aspect, the present disclosure provides a set of metrics for evaluating entities of a data source, the set of metrics comprising: at least one overlap between a plurality of predictions; a set of top correlations of objects in a database; a set of top processes; at least one correlation of the predictions with metadata associated with database objects; a proportion of the predictions derived from ligandable drug target families; a percentage of processes or pathways found in an enrichment of gene data in a training model and in enriched lists of the plurality of predictions; at least one overlap between pathway enrichment or process enrichment data between the entities, a summary of relationships associated with the predictions to one or more objects in a database; at least one reduction to practice statement of association between the plurality of predictions and a disease context; and at least one connectivity associated with protein-protein interactions.

In a third aspect, the present disclosure provides a system for comparing and evaluating a plurality of predictions based on a set of metrics, the system comprising: an input module configured to receive one or more sets of entities and associated metadata from a data source; a processing module configured to predict, based said one or more sets of entities in relation to the data source, the plurality of predictions, wherein the plurality of predictions are ranked in a subset set of predictions; a computation module configured to compute the set of metrics based on the plurality of prediction and the associated metadata, wherein the computation is performed using one or more pre-trained predictive models; and an output module configured to present the set of metrics for evaluation.

In a fourth aspect, the present disclosure provides an interface device for displaying a set of metrics, the interface device comprising: a memory; at least one processor configured to access the memory and perform operations according to any of above aspects; an output model configured to output the set of metrics; and an interface configured to display at least one display option comprising: an overlap option, a top pathways option, a model-literature option, a ligandability option, a mistake targets option, a pathway enrichment option, a process enrichment option, a disease pathway recall option, a disease process recall option, a disease benchmark interactions option, a reduction to practice presence option, and a protein-protein interaction connectivity option.

The methods described herein may be performed by software in machine-readable form on a tangible storage medium e.g. in the form of a computer program comprising computer program code means adapted to perform all the steps of any of the methods described herein when the program is run on a computer and where the computer program may be embodied on a computer-readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

This application acknowledges that firmware and software can be valuable, separately tradable commodities. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

The preferred features may be combined as appropriate, as would be apparent to a skilled person, and may be combined with any of the aspects of the invention.

### BRIEF DESCRIPTION OF THE DRAWINGS

Embodiments of the invention will be described, by way of example, with reference to the following drawings, in which:

FIG. 1 is a flow diagram illustrating an example process of generating a set of metrics for comparing entities of a knowledge graph according to the invention;

FIG. 2a is a flow diagram illustrating another example process of generating the set of metrics to be displayed through an interface device according to the invention;

FIG. 2b is a flow diagram illustrating yet another example process of generating the set of metrics where an application module is configured to communicate the set of metrics externally through the application module according to the invention;

FIG. 3 is a schematic illustrating another example process of generating a plurality of predictions from different pre-trained predictive models according to the invention;

FIG. 4a is a schematic diagram illustrating another example of the set of metrics as display options presented on the interface according to the invention;

FIG. 4b is a schematic diagram illustrating another example in relation to FIG. 4a of the set of metrics as display options presented on the interface according to the invention;

FIG. 4c is a schematic diagram illustrating another example in relation to FIGS. 4a and 4b of the set of metrics as display options presented on the interface according to the invention;

FIG. 5 is a schematic diagram of a unit example of a subgraph of the knowledge graph applicable to FIGS. 1 to 4b; and

FIG. 6 is a schematic diagram of a computing device suitable for implementing embodiments of the invention.

Common reference numerals are used throughout the figures to indicate similar features.

### DETAILED DESCRIPTION

Embodiments of the present invention are described below by way of example only. These examples represent the suitable modes of putting the invention into practise that are currently known to the applicant, although they are not the only ways in which this could be achieved. The description sets forth the functions of the example and the sequence of steps for constructing and operating the example. However, the same or equivalent functions and sequences may be accomplished by different examples.

Herein disclosed is at least a method to generate metrics or a set that aids a user in evaluating and comparing entities to be used in a predictive machine learning model. In this method, a user selects the entities—either individual or grouped—from a data source that they wish to compare. Predictive models are run for each entity or group, and the top N predictions based on relationships in the knowledge graph are extracted. Further metadata relating to the entities and the predicted targets is extracted from the knowledge graph and combined with data from the predictions. All this data is run through a series of calculations in order to produce the evaluation set of metrics based on the top predictions and metadata associated with each entity or group. Finally, the set of metrics are output in a user interface so that a user is able to evaluate a broad overview of the outputs that using each entity (or group of entities) in a predictive model would generate so as to determine the preferable entity to use.

Accordingly, employing the set of metrics generated enables a user to efficiently compare the impact that using different input entities has on a model or decide which entities produce the most useful results. Moreover, the decision process may be an iterative process achieved through deploying one or more predictive machine learning (ML) models or ML-based model together with or without the user.

ML model(s), predictive algorithms and/or techniques may be used to generate a trained model such as, without limitation, for example one or more trained ML models or classifiers based on input data referred to as training or annotated data associated with ‘known’ entities and/or entity types and/or relationships therebetween derived from large scale datasets (e.g. a corpus or set of text/documents or unstructured data). The input data may also include graph-based statistics as described in more detail in the following sections. With correctly annotated training datasets in such fields as, without limitation, for example chem(o)informatics and bioinformatics, techniques can be used to generate further trained ML models, classifiers, and/or analytical models for use in downstream processes such as, by way of example but not limited to, drug discovery, identification, and optimisation and other related biomedical products, treatment, analysis and/or modelling in the informatics, chem(o)informatics and/or bioinformatics fields. The term ML model is used herein to refer to any type of model, algorithm or classifier that is generated using a training data set and one or more ML techniques/algorithms and the like.

Examples of ML model/technique(s), structure(s) or algorithm(s) that may be used by the invention as described herein may include or be based on, by way of example only but is not limited to, one or more of: any ML technique or algorithm/method that can be used to generate a trained model based on a labelled and/or unlabelled training datasets; one or more supervised ML techniques; semi-supervised ML techniques; unsupervised ML techniques; linear and/or non-linear ML techniques; ML techniques associated with classification; ML techniques associated with regression and the like and/or combinations thereof. Some examples of ML techniques/model structures may include or be based on, by way of example only but is not limited to, one or more of active learning, multitask learning, transfer learning, neural message parsing, one-shot learning, dimensionality reduction, decision tree learning, association rule learning, similarity learning, data mining algorithms/methods, artificial neural networks (NNs), autoencoder/decoder structures, deep NNs, deep learning, deep learning ANNs, inductive logic programming, support vector machines (SVMs), sparse dictionary learning, clustering, Bayesian networks, types of reinforcement learning, representation learning, similarity and metric learning, sparse dictionary learning, genetic algorithms, rule-based machine learning, learning classifier systems, and/or one or more combinations thereof and the like.

In relation to ML model/technique(s), structure(s) or algorithm(s) is the annotated or labelled dataset(s) for the training of the above; the training data may include but are not limited to, for example, the data corresponding to entities of interest associated with entities such that of diseases, biological processes, pathways and potential therapeutic targets. The data corresponding to the entities of interest may be extracted from various structured and unstructured data sources, and literature via natural language processing or other data mining techniques.

For entity evaluation whether by the user or an ML model, the set of generated metrics include: at least one overlap between a plurality of predictions; a set of top correlations of objects in a database or relations to other objects in the database, where the set of top correlation may be a set of top pathways; at least one correlation of the predictions with metadata associated with database objects or correlation of prediction scores with any other metadata values from the database, where the at least one correlation may be a prediction using literature evidence; a proportion of the predictions derived from ligandable drug target families; a percentage of processes or pathways found in an enrichment of gene data in a training model and in enriched lists of the plurality of predictions; at least one overlap between pathway enrichment or process enrichment data between the entities, a summary of relationships associated with the predictions to one or more objects in a database or measurement of particular relationship from the prediction to be one or more object in the database, wherein the summary or measurement may be at least one disease benchmark interaction; at least one reduction to practice statement of association between the plurality of predictions and a disease context; and at least one connectivity associated with protein-protein interactions.

Any one or more of the above set of metrics may be used for the overall entity evaluation or to determine whether one entity from a data source is superior over another in the selection or optimisation process. The data source may be a knowledge graph. In addition to or in place of the knowledge graph, other data sources may be used such as a Query Language (SQL) server, or file structure for storing relational data formatted in Comma Separated Values (CSV), or any other suitable relational databases.

More specifically, each metric is designed to capture relevant characteristics of predictions based on the concerns of a user and to bolster target identification and/or the likelihood of success during experimentation. Such concerns may be related to factors such as disease relevance, safety, and druggability. In turn, the metric or the set of metrics described herein effectively assess and compare the suitability of the initial entities or which entities produce the most useful results given the model. This may be done without further model evaluation.

For example, in considering a factor such as a disease relevance, it can be understood that an assessment of disease relevance may be accomplished via employing one or more metrics, that is, by measuring how much the predicted gene targets interact biologically (via PPI or protein-protein interaction) with a set of well know disease gene targets. In this example, a summary of relationships associated with the predictions of objects may be established specifically by benchmarking disease interactions using packages and databases such as Signor, Omnipath, Kegg, and Biogrid. In addition, connectivity associated with protein-protein interaction may be assessed or evaluated

The disease benchmark interactions metric helps a user to select entities for which the predicted targets will modulate the benchmark targets for the disease, where an entity with high disease benchmark interactions is more desirable. This is done by calculating the proportion of the disease benchmark that interacts directly with the prediction list targets via PPI edges or by way of measuring connectivity associated with PPI.

For two predictions A and B, prediction A may interact biologically with 23% of the disease benchmark set while prediction B interacts with 57% of the disease benchmark set. It is thereby indicative that prediction B is more disease-relevant than prediction A based on this metric.

Alternative or additional metrics for the set may be employed together with the metric for providing the summary of relationships in order to determine whether to accept prediction A over B.

Another metric is for evaluating the amount of overlap between a plurality or a list of predictions. The list of overlaps provides a measure of how similar the different target prediction lists may be. It achieves this by calculating the percentage of overlap between the lists. Furthermore, it may list the top, i.e. 20, overlapping and non-overlapping targets, where overlapping targets are those that are predicted for more than one of the initial entities.

Another metric is related to assessing a set of top correlations of objects in a database. An example of the assessment may be the evaluation of top, i.e. 10, biological pathways. In this example, the top pathways can provide a better understanding of whether the target list is enriched for mechanisms that are relevant and specific to the disease of interest, this time by examining the enrichment of Reactome pathways. Again using the top 200 targets, the metric calculates the enrichment of Reactome pathways using the Fisher exact test and corrects for multiple testing. The list is filtered by the FDR-adjusted p-value of the Fisher exact test and sorted by the odds ratio.

Another metric, similar to the evaluation of top pathways, is assessing a set of top processes associated. This metric allows a better understanding of whether the target list is enriched for processes that are important to the disease entity of interest. The metric calculates, based on the top targets, the enrichment of Gene Ontology (GO) processes using the Fisher exact test and correcting for multiple testing. The list is sorted by the FDR-adjusted p-value of the Fisher exact test.

Another metric or a combination of two or more metrics for process recall from training data. By doing so, this metric or metrics help assess whether the selected entities, for which the predicted targets, will modulate the GO processes linked to the disease biology. The enrichment of GO Processes uses the top targets for ensuing calculation via the Fisher exact test, and the calculated results are corrected for multiple testing. Using a data source such as a knowledge graph, the GO processes enriched in the disease training data are then retrieved. An intersection of the above two lists is calculated as a percentage of the GO processes enriched in the disease training data. Effectively, a percentage of such processes or pathways found in the enrichment of gene data in a training model and in enriched lists of the plurality of predictions is thereby determined, and thus provide a determination of overlap between pathway enrichment or to process enrichment data between the entities.

Another metric or a combination of two or more metrics may ascribe to selecting for popular targets. Target predictions that appear frequently, or are deemed popular, because they are linked to many diseases are highlighted. Due to the frequency of appearance of these highlights, targets are consistently rejected in triage. The purpose here is to help judge whether the selected initial entities cause the predictive models to generate targets that are specific to the disease as opposed to these common targets.

In terms of target specificity, an assessment of how specific a target is to other diseases is performed. It calculates the number of diseases that each target is linked to via the disease benchmark or training data and then calculates the log-adjusted mean number of connected diseases for the top targets. By using benchmark data, it also allows a user to assess if the models are reasoning through PPI edges to benchmark targets instead of merely selecting frequently occurring targets.

In effect, correlations of the predictions with metadata (any of which associated with entities and the predicted targets is extracted from a data source) associated with the data source objects may be evaluated, specifically by identifying the most popular targets in accordance with literature evidence or obtaining underlying correlations. Then the quantity and rank of the targets are calculated and produced from the selected prediction lists or across the benchmark entities. The results provide the basis for further prediction evaluation. As such, the correlations of the predictions may also be evaluated in combination with the following metric or metrics.

Another metric is related to the reduction to practice (RTP) statement of association between the plurality of predictions and a disease context. RTP statements or sentences indicate a target has been modulated to impact a disease phenotype in a disease model. This metric calculates the percentage of the prediction list with at least one RTP connection to the disease, allowing the evaluation of the targets in the context of the disease.

Another metric or a combination of two or more metrics is related to capturing model predictions' correlation with counts of articles with syntactically linked pairs (SLP) between the initial entities and targets. In other words, to perform an evaluation using model score or SLP count correlations. SLPs have high recall and allow users to assess the level of evidence between a target and a disease through the article count. High correlations might suggest predictions are closely aligned to the existing literature evidence, while low correlations could indicate a lack of capturing important biology. In this case, not only may the proportion of predictions derived from ligandable drug target families be evaluated, but also provides an implicit assessment with the connectivity associated with any protein-protein interaction.

It can be determined whether the initially selected entities cause the models to predict targets of a particular protein class as opposed to simply re-ranking the druggable genome for each deployment. This is accomplished by capturing the distribution of target protein classes, i.e. Kinases, TFs, GPCRs, Enzymes, Transporters, and Unknowns, in the form of percentages.

Although details of the present disclosure may be described, by way of example only but are not limited to, with respect to biomedical, biological, chem(o)informatics or bioinformatics entities, presented or stored in the form of knowledge graphs or other appropriate data structures, are to be appreciated by the skilled person that the details of the present disclosure are applicable as the application demands to any other type of entity, information, data informatics fields and the like. For example, the ML models or metrics described above can be applied to any of any other type of entity, information, data informatics fields and the like insofar described in the present disclosure.

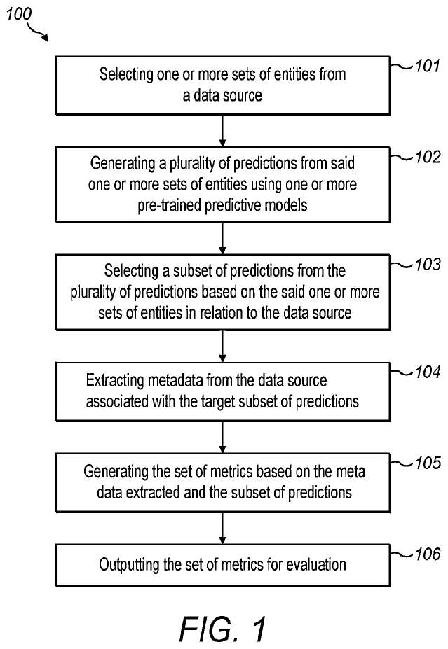

FIG. 1 is a flow diagram illustrating an example process 100 of generating a set of metrics for comparing entities. One or more sets of entities are selected from a data source. A plurality of predictions aggregated from said one or more sets of entities using one or more pre-trained predictive models is generated. A subset of predictions is selected from the plurality of predictions based on the said one or more sets of entities in relation to the knowledge graph. Metadata is extracted associated with the subset of predictions and used to generate the set of metrics. The set of metrics is outputted for evaluation.

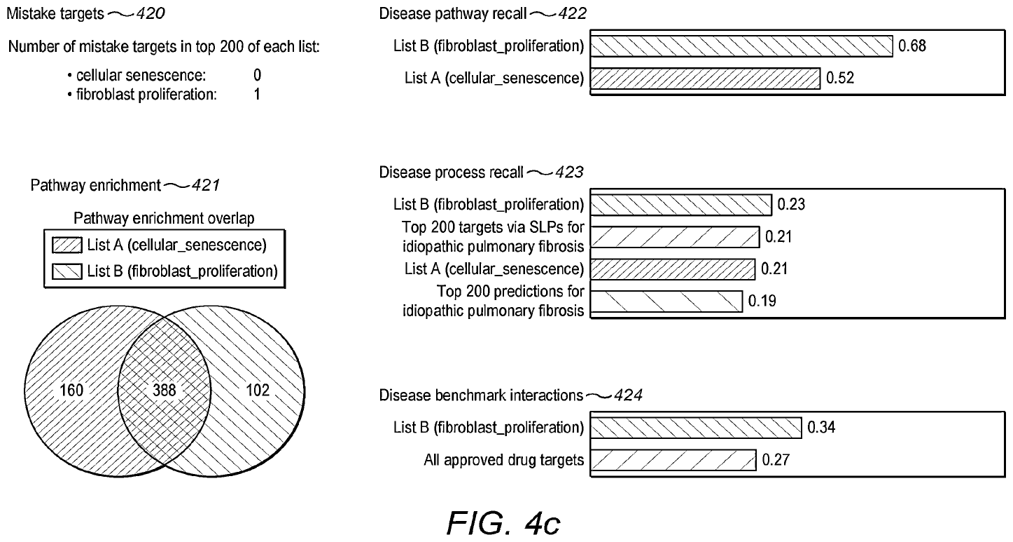

In step 101, one or more sets of entities are elected. The selection is from a data source, for example, a knowledge graph or a subgraph as depicted in FIG. 5. The selection of the entities may also be from one or more combinations of data sources, including the knowledge graph. Another source may be SQL, CSV, or any other relational database. In the case that a knowledge graph is the source, the knowledge graph may be configured to encode data related to the biomedical domain or a field corresponding to various domains, for example, a biomedical domain.

In step 102, generating a plurality of predictions aggregated from said one or more sets of entities using one or more pre-trained predictive models; the subset of predictions may comprise top predictions ranked in relation to said one or more pre-trained predictive models. The top predictions may comprise predictions with the best predictive scores (or metrics for scoring the predictions comparatively) selected from the entire set of predictions. The predictive score or metrics may be generated via the pre-trained predictive models. Each pre-trained predictive model is configured to generate predictive scores that are compatible for evaluating the best predictive score in the event that two or more predictive models are used. The predictive scores may also be derived externally using the predictive models. The one or more pre-trained predictive models may also be adapted for a biomedical context, that is the one or more pre-trained predictive models are trained using biomedical data. This biomedical data may be enriched. The data may also undergo a process of enrichment, for example, using data further extracted from multiple sources.

The one or more pre-trained predictive model(s) may comprise any one or more of the ML model(s) herein described. The one or more pre-trained predictive model(s) may also be one or customised models such as Distributions over Latent Policies for Hypothesizing in Networks (DOLPHIN) disclosed in and with reference to U.S. provisional application 63/086,903, Graph Pattern Inference disclosed in and with reference to U.S. provisional application 63/058,845, Graph Convolutional Neural Network (GCNN) disclosed in and with reference to U.S. provisional application 62/673,554. Other models include examples such as Rosalind, published according to Paliwal, S., de Giorgio, A., Neil, D. et al. “Preclinical validation of therapeutic targets predicted by tensor factorization on heterogeneous graphs.” Sci Rep 10, 18250 (2020) (https://doi.org/10.1038/s41598-020-74922-z). These models are intended to produce different results. The models may be aggregated differently. One way to aggregate may be to apply an interleaving approach that takes the top targets from each model and the top consensus predictions across the models.

In step 103, selecting a subset of predictions from the plurality of predictions based on the said one or more sets of entities in relation to the data source; the data source may be a knowledge graph. The selected subset of predictions may be top predictions from the knowledge graph or any other data sources. The subset of predictions establishes the basis for the metrics generation in step 105.

In step 104, extracting metadata associated with the subset of predictions; the metadata comprises entity metadata and predicted metadata. These metadata are associated with each entity group. Together with the subset of predictions, the associated metadata may be used to generate the set of metrics as in step 105, where the set of metrics is generated based on the metadata extracted and the subset of predictions.

More specifically, the set of metrics may be generated based on predictions and associated metadata. The associated metadata, in this case, may comprise the predicted metadata.

The generated set of metrics may comprise or based on one or a combination of: overlap between the plurality of predictions, set top correlations of objects in a database, set of top processes, correlation of the predictions with metadata associated with database objects, proportion of predictions derived from ligandable drug target families, percentage of processes or pathways found in an enrichment of gene data in a training model and in enriched lists of the plurality of predictions, overlap between pathway enrichment or process enrichment data between the entities, summary of relationships associated with the predictions to one or more objects in a database, reduction to practice statement of association between the plurality of predictions and a disease context, and connectivity associated with protein-protein interactions.

In step 105, outputting the set of metrics for evaluation. The output may be displayed on an interface. The interface may comprise one or more display options configured to display one or more herein described metrics or based on one or more metrics. The interface may be a device that is configured to receive one or more inputs of entities associated with a data source such as a knowledge graph.

The outputted set of metrics may be evaluated with at least one automated system. The automated system may be configured to process or select one or more predictions based on at least one predetermined criterion associated with the outputted set of metrics. The automated system may be associated with the predictive machine learning model. The entities of the data source may be further evaluated based on the outputted set of metrics.

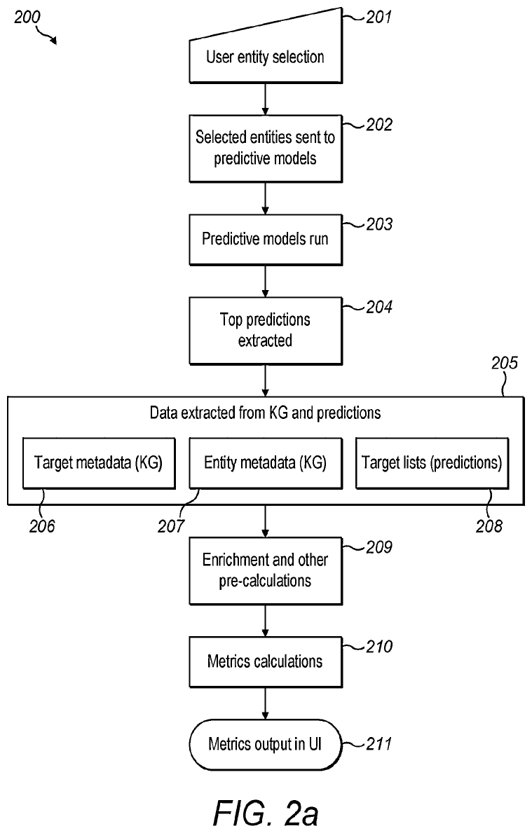

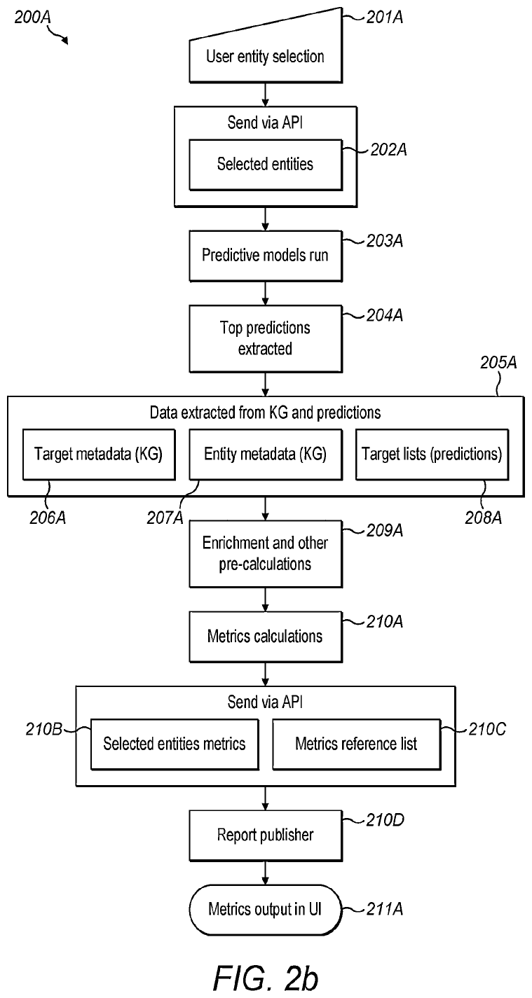

FIG. 2a is a flow diagram illustrating another example process 200 of generating the set of metrics to be displayed through an interface device. The method starts with a user or automated system selecting from a knowledge graph the entities for which comparison metrics are to be generated 201.

For example, these entities may include individual entities, or a group of entities clustered together. In the context of a biomedical application, for example, a user may wish to examine the genes, treatments, and processes associated with type 2 diabetes in order to formulate a better understanding of the disease and how to treat it. To do this, the user might compare the singular type 2 diabetes entity with a group of entities that contains—for instance—type 2 diabetes and several closely related entities such as type 2 diabetes complications, type 2 diabetes onset, and type 2 diabetes subtype.

Once selected, entities may be sent to one or more pre-trained predictive machine learning models 202. The predictive models run for each entity or group of entities 203. Predictive models may thus be any algorithms that generate predicted relationships between entities in a data source, based on factors such as similar extant relationships. Multiple different types of predictive models can be run for each entity or group such that multiple sets of target predictions are generated. The entities that are predicted to be connected to the initial entities are referred to as targets. In the context of the data source being a biomedical knowledge graph, if the initial entities selected represent a disease, the predicted target entities may represent genes or processes that are causally linked to the disease.

Target predictions are output by the predictive models and aggregated so that the top N predictions for each entity or group can be selected 204. These top predictions will be the basis for the metrics calculations. Sampling is used rather than the entire prediction dataset in order to capture and exaggerate the difference between the datasets associated with each initial entity or group. This has the further benefit of being less time consuming than if the metrics were to be generated for the entire predictions dataset and so a more streamlined user experience is possible. In practice, it has been found that the top 200 predictions provide a suitable level of clarity, though his number can be adjusted as appropriate.

Additional metadata is extracted from the knowledge graph and combined with data from the target predictions 205. This data is composed of: metadata associated with the target predictions 206; metadata associated with the selected entities 207; and lists of the targets 208. This data provides context surrounding the initial entities and target predictions which contributes to the metrics calculations. Metadata may include data extracted from unstructured sources. For example, in a biomedical context, it might include RTP sentences which signify proven therapeutic or biological relationships.

This data may be enriched, and other pre-calculations could run 209 in order to prepare the data that the metric calculations may be run over it 210. Enrichment is the process of further complementing the datasets with data extracted from other sources. For example, in a biomedical context, enrichment using a combination of structured databases—for instance, Reactome, Gene Ontology, and CTD—and proprietary unstructured data from research papers may provide a suitable level of detail. The metrics used may vary in order to best suit the models used and field of knowledge, but examples that would likely prove useful across multiple fields include: finding the overlap between the prediction lists for each set of entities; calculations of which target predictions frequently appear in a specific field of knowledge and so whose presence is less informative; the extent to which the models' predictions correlate with SLP in literature.

The calculated metrics are output in a user interface 211 for a user or an automated system to evaluate the suitability of their initially selected entities for the task they wish to perform.

FIG. 2b is a flow diagram illustrating yet another example process 200A of generating the set of metrics in accordance with FIG. 2a, where an application module is configured to communicate the set of metrics externally through the application module. In FIG. 2b, the generation of the set of metrics is the same as presented in FIG. 2a. That is, reference numeral 201A, 202A, 203A, 204A, 205A, 206A, 207A, 208A, 209A, 210A, 21A of FIG. 2b correspond to 201 to 211 of FIG. 2a respectively.

In addition, in FIG. 2b, the user selects entities or entity groups in a user interface 201A, and this selection 202A is communicated via an API, to a separate software programme comprising the pre-trained models to be run.

After metrics have been calculated 210A, the output metrics for each entity or group 211B and a reference list of metrics 212C are set via an API to a report publisher 210D. The report publisher 210D collates the metrics data and compiles a report that explains and visualises the metrics for user consumption in a user interface 211A. In response to receiving said one or more inputs and following the output of the set of metrics, an external application module may be configured to receive the outputted set of metrics and an associated metrics reference list from said at least one processor of the user interface 211A or an interface device.

In addition, a second application module may be configured to receive the outputted set of metrics and the associated metrics reference list for a report publisher 210D. In this case, the report publisher 210D may be configured to collate and compile the received set of metrics and the associated metrics reference list to generate a representative report for visualising the set of metrics as display options on the interface device.

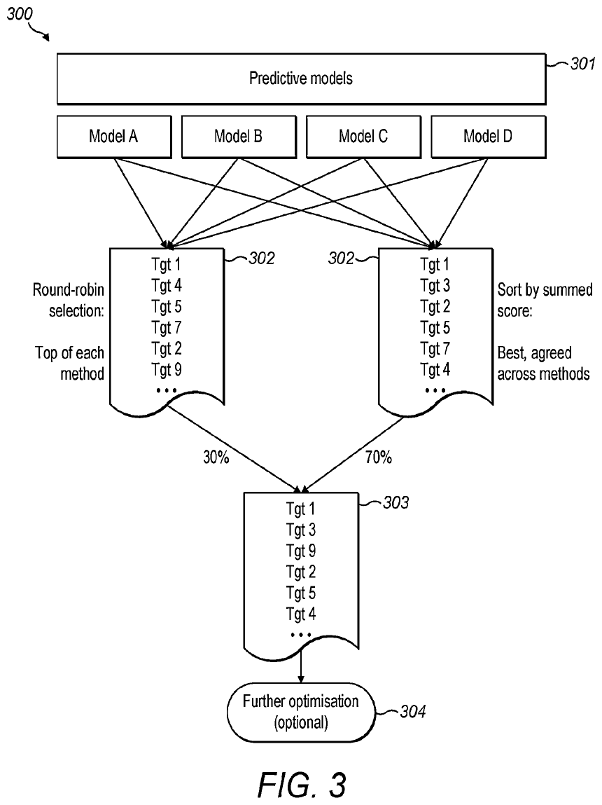

FIG. 3 is a schematic illustrating another example process 300 for generating a plurality of predictions from different pre-trained predictive models; the figure outlines predictive models A, B, C, and D, with each model directed to one or more list of selections. The list selects are then aggregated and appropriately weighted to form a master or optimal list. Here, targets 1, 4, 5, 7, 2, and 9 from the left list and targets 1, 3, 2, 5, 7, and 4 from right list combined to produce a list comprising targets 1, 3, 9, 2, 5, and 4. The weighting ratio are 3:7 respectively for left and right lists.

FIG. 3 therefore provides an overview of the method used to aggregate target predictions utilising a range of predictive models or their combination. In a biomedical context, this combination may comprise omics-based models and knowledge graph models. The exemplar embodiment shown in FIG. 3 uses four predictive models 301. Specifically, the target predictions from all the predictive models are listed together. The colour coding used indicates this merging of predictions. The list is duplicated and ranked twice 302 once using a round-robin selection technique, and once using the sum of the targets' scores from across all predictive models—before the two target rankings are recombined with appropriate weighting 303. The top targets could be taken from this list, or the lists could be further optimised to favour certain features 304. In one aspect, further optimisation with an ML-based method for predicting annotations may be introduced. The drug discovery experts may help annotate whether a potential drug target is likely to be progressible or non-progressable in relation to the ML-based method.

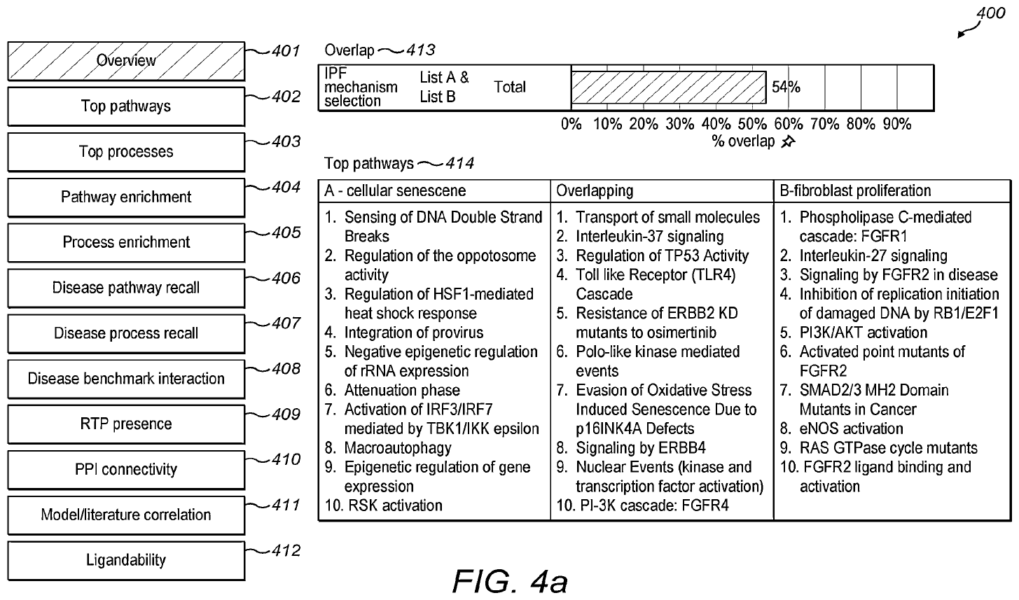

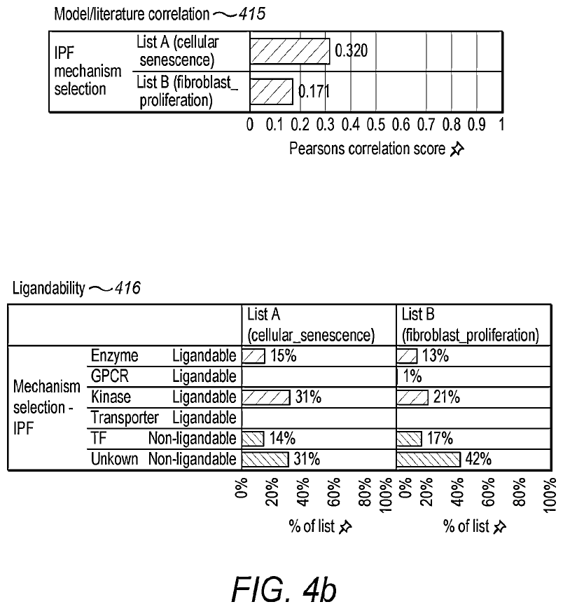

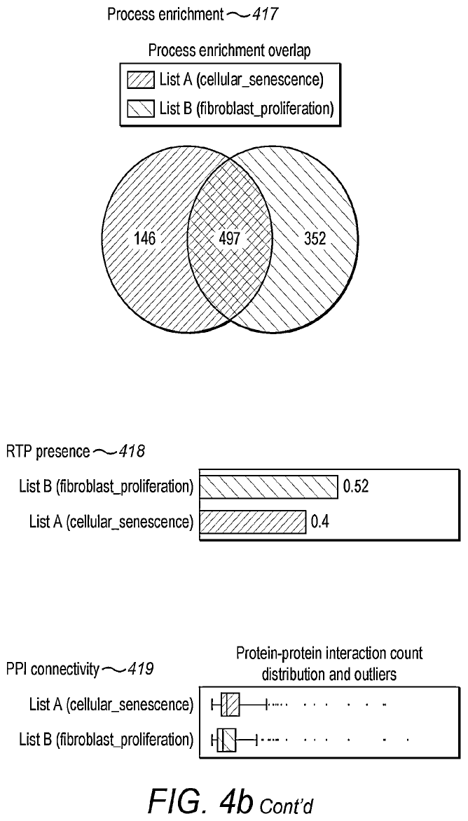

FIGS. 4a to 4c are schematic diagrams illustrating another example of the set of metrics 400. The set of metrics may be used to aid in entity selection for drug target prediction or used in another biomedical context. The selected entities under review may either be diseases or mechanisms, while the predicted target entities may be genes or processes that have close causal links with the disease under review. Predictive models and one or more data sources may be used to generate these set of metrics such as those specific to the biomedical field. The set of metrics may be outputted onto a user interface. An example of a user interface and the underlying set of metrics may be depicted accordingly.

In the FIGS. 4a to 4c is a list of display options shown and separated as tabs. The display options include an overlap option, a top pathways option, a model-literature option, a ligandability option, a mistake targets option, a pathway enrichment option, a process enrichment option, a disease pathway recall option, a disease process recall option, a disease benchmark interactions option, a reduction to practice presence option, and a protein-protein interaction connectivity option. These display options are related to the set of metrics.

Also related to the set of metrics are display tabs shown in FIG. 4a, where each tab is associated with a display option. The tabs may include tabs for top pathways 402, top processes 403, pathway enrichment 404, process enrichment 405, disease pathway recall 406, disease process recall 407, disease benchmark interaction 408, RTP presence 409, PPI connectivity 410, model/literature correlation 411, and ligandability 412. The tabs are categorized under or displayed with an overview tab 401. These tabs may be displayed in a manner suitable on an interface device or interface. The tabs may provide examples of how a user may interact with the various display options, as shown in FIGS. 4a to 4c.

In another example, also shown in FIG. 4a, the overlap option displays 413 a percentage of 54% for A and B lists in relation to IPF mechanism selection. The A and B lists represent cellular senescence and fibroblast proliferation, respectively. For the top pathway option 414, it is shown that A list or representing cellular senescence (1. Sensing of DNA Double Strand Breaks, 2. Regulation of the apoptosome activity, 3. Regulation of HSF1-mediated heat shock response, 4. Integration of provirus, 5. Negative epigenetic regulation of rRNA expression, 6. Attenuation phase, 7. Activation of IRF3/IRF7 mediated by TBK1/IKK epsilon, 8. Macroautophagy, 9. Epigenetic regulation of gene expression, and 10. RSK activation) and with B list or representing fibroblast proliferation (1. Phospholipase C-mediated cascade: FGFR1, 2. Interleukin-27 signaling, 3. Signaling by FGFR2 in disease, 4. Inhibition of replication initiation of damaged DNA by RB1/E2F1, 5. PI3K/AKT activation, 6. Activated point mutants of FGFR2, 7. SMAD2/3 MH2 Domain Mutants in Cancer, 8. eNOS activation, 9. RAS GTPase cycle mutants, and 10. FGFR2 ligand binding and activation). In the middle is the Overlapping list (1. Transport of small molecules, 2. Interleukin-37 signalling, 3. Regulation of TP53 Activity, 4. Toll-like receptor 4 (TLR4) cascade, 5. Resistance of ERBB2 KD mutants to osimertinib, 6. Polo-like kinase mediated events, 7. Evasion of Oxidative stress Induced Senescence Due to p 16INK4A Defects, 8. Signaling by ERBB4, 9. Nuclear Events (kinase and transcription factor activation), and 10. PI-3K cascade:FGFR4).

Further to this example, shown in FIG. 4b are display options for model-literature correlation 415, ligandability 416, process enrichment 417, RTP presence 418, and PPI connectivity 419. In each of these options, A and B lists are compared and displayed accordingly. It is shown for model-literature option 415 ranges between 0 to 1 that A list has a Pearson score of 0.320, and B list has a score of 0.171. It is shown for ligandability 416 with respect to both ligandable and non-ligandable protein classes. These classes include Enzyme, GPCR, Kinases, Transporters, TF, and remaining classing as unknown. The classes specified by a range of percentages. For Enzyme class 15% to 13% is shown respectively for A and B lists; GPCR class 0% and 1%; Kinase class 31% to 21%; Transporter class 0% to 0%; TF class 14% to 17%; and finally unknown class 31% to 41%. It is shown for process enrichment 417 in a van diagram that 146 for A list and 352 for B list together with 497 overlapping both lists. It is shown for RTP presence option 418 that A list is 0.52 while B list is only 0.4. It is shown for PPI connectivity option 419 with respect to protein-protein interaction count distribution and outliers that help distinguish between A and B lists.

Again in the example, in FIG. 4c are display options for mistake targets 420, pathway enrichment 421, disease pathway recall 422, and disease benchmark interactions 423. It is shown for mistaken targets option 420 that a top 200 list is taken into consideration. The number of mistake targets in this list of 200 is only a single case of B list. It is shown for pathway enrichment option 421 similarly as process enrichment by a van diagram that 160 for A list and 102 for B list together with 388 overlapping both lists. It is shown for disease pathway recall option 422 that B list, 0.68 is greater than A list, 0.52. It is shown for disease process recall option 423 that B list, 0.21 is less than A list, 0.23. For the same, but with regards to a top 200 targets via SLPs for idiopathic pulmonary fibrosis, B list, 0.19 is relatively close to A list, 0.20. Finally, it is shown for disease benchmark interactions option 424 that B list, 0.34 is greater than A list 0.24. The all approved drug target sits at 0.27 between both lists.

The above-described display options, shown and exemplified in FIGS. 4a to 4c, may be part of an interface device. The interface device may further be configured to receive one or more inputs of entities associated with a data source. In response to receiving said one or more inputs and following the output of the generated set of metrics, there may be an external application module or as an API. The external application module or API may be configured to receive the outputted set of metrics and an associated metrics reference list from said at least one processor of the interface device.

The interface device for displaying the display options may further include a second application module. This model may be configured to receive the outputted set of metrics and the associated metrics reference list for a report publisher. The report publisher may be configured to collate and compile the received set of metrics and the associated metrics reference list to generate a representative report for visualising the set of metrics as display options on the interface device in a suitable format, for example, shown in FIGS. 4a to 4c.

FIG. 5 is a schematic diagram of a unit example of a subgraph 500 of the knowledge graph applicable to FIGS. 1 to 4c; the figure shows an example of a small knowledge graph, with nodes representing entities and edges representing relationships. An entity 501 may be linked to another entity 503 by an edge 502, the edge being labelled with the form of the relationship. For example, in the biomedical domain, the first entity may be a gene and the second may be a disease. Thus, the edge would represent a gene—disease relationship, which may be tantamount to “causes” if the gene is responsible for the presence of the disease.

Expanding on this example, if the third entity 504 was a disease and shared a disease—disease relationship 505 with Entity 2, a new gene-disease edge between Entity 1 and Entity 2 506 may be inferred by a predictive model examining a data model configured to include the knowledge graph depicted in the figure. However, these inferences may not always prove to be correct. Thus, a predictive model may score the likelihood of an inferred link, and these scores can contribute to ranking target entities.

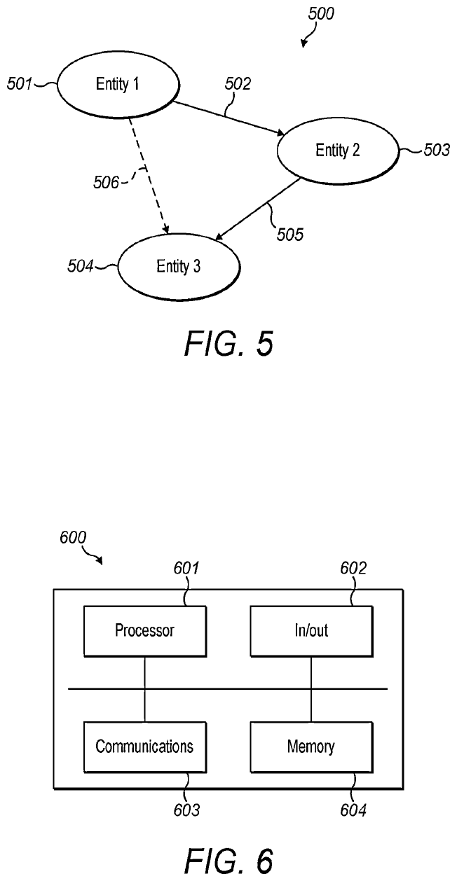

FIG. 6 is a schematic diagram illustrating an example computing apparatus/system 600 that may be used to implement one or more aspects of the system(s), apparatus, method(s), and/or process(es) combinations thereof, modifications thereof, and/or as described with reference to FIGS. 1 to 5 and/or as described herein. Computing apparatus/system 600 includes one or more processor unit(s) 601, an input/output unit 602, communications unit/interface 603, a memory unit 604 in which the one or more processor unit(s) 601 are connected to the input/output unit 602, communications unit/interface 603, and the memory unit 604. In some embodiments, the computing apparatus/system 600 may be a server, or one or more servers networked together. In some embodiments, the computing apparatus/system 400 may be a computer or supercomputer/processing facility or hardware/software suitable for processing or performing the one or more aspects of the system(s), apparatus, method(s), and/or process(es) combinations thereof, modifications thereof, and/or as described with reference to FIGS. 1 to 5 and/or as described herein. The communications interface 403 may connect the computing apparatus/system 600, via a communication network, with one or more services, devices, the server system(s), cloud-based platforms, systems for implementing subject-matter databases and/or knowledge graphs for implementing the invention as described herein. The memory unit 604 may store one or more program instructions, code or components such as, by way of example only but not limited to, an operating system and/or code/component(s) associated with the process(es)/method(s) as described with reference to FIGS. 1 to 5, additional data, applications, application firmware/software and/or further program instructions, code and/or components associated with implementing the functionality and/or one or more function(s) or functionality associated with one or more of the method(s) and/or process(es) of the device, service and/or server(s) hosting the process(es)/method(s)/system(s), apparatus, mechanisms and/or system(s)/platforms/architectures for implementing the invention as described herein, combinations thereof, modifications thereof, and/or as described with reference to at least one of the FIGS. 1 to 5.

With regards to the above figures, in one aspect is a computer-implemented method of generating a set of metrics for evaluating entities used with a predictive machine learning model, the method comprising: selecting one or more sets of entities from a data source; generating a plurality of predictions aggregated from said one or more sets of entities using one or more pre-trained predictive models; selecting a subset of predictions from the plurality of predictions based on said one or more sets of entities in relation to the data source; extracting metadata from the data source associated with the subset of predictions, wherein the metadata comprises entity metadata and predicted metadata; generating the set of metrics based on the metadata extracted and the subset of predictions; and outputting the set of metrics for evaluation.

In another aspect is set of metrics for evaluating entities of a data source, the set of metrics comprising: at least one overlap between a plurality of predictions; a set of top correlations of objects in a database; a set of top processes; at least one correlation of the predictions with metadata associated with database objects; a proportion of the predictions derived from ligandable drug target families; a percentage of processes or pathways found in an enrichment of gene data in a training model and in enriched lists of the plurality of predictions; at least one overlap between pathway enrichment or process enrichment data between the entities, a summary of relationships associated with the predictions to one or more objects in a database; at least one reduction to practice statement of association between the plurality of predictions and a disease context; and at least one connectivity associated with protein-protein interactions.

In another aspect is a system for comparing and evaluating a plurality of predictions based on a set of metrics, the system comprising: an input module configured to receive one or more sets of entities and associated metadata from a data source; a processing module configured to predict, based said one or more sets of entities in relation to the data source, the plurality of predictions, wherein the plurality of predictions are ranked in a subset set of predictions; a computation module configured to compute the set of metrics based on the plurality of prediction and the associated metadata, wherein the computation is performed using one or more pre-trained predictive models; and an output module configured to present the set of metrics for evaluation.

In another aspect is an interface device for displaying a set of metrics, the interface device comprising: a memory; at least one processor configured to access the memory and perform operations according to any of above aspects; an output model configured to output the set of metrics; and an interface configured to display at least one display option comprising: an overlap option, a top pathways option, a model-literature option, a ligandability option, a mistake targets option, a pathway enrichment option, a process enrichment option, a disease pathway recall option, a disease process recall option, a disease benchmark interactions option, a reduction to practice presence option, and a protein-protein interaction connectivity option.

In another aspect is a computer-readable medium storing code that, when executed by a computer, causes the computer to perform the computer-implemented method or to process the set of metrics of any above aspects.

As an option, the subset of predictions comprises top predictions ranked in relation to said one or more pre-trained predictive models.

As another option, said one or more pre-trained predictive models are adapted for a biomedical context.

As another option, said one or more pre-trained predictive models are trained using biomedical data.

As another option, said biomedical data is enriched or has undergone a process of enrichment using data further extracted from one or more sources.

As another option, the set of metrics are generated based on said top predictions and associated metadata.

As another option, said associated metadata comprising said predicted metadata.

As another option, selecting said one or more set of entities from the data source that comprises a knowledge graph; and extracting metadata from the knowledge graph, wherein the knowledge graph is configured to encode data related to the biomedical domain or a field corresponding to the biomedical domain.

As another option, the set of metrics are based on one or a combination of: at least one overlap between the plurality of predictions, a set top correlations of objects in a database, a set of top processes, at least one correlation of the predictions with metadata associated with database objects, a proportion of the predictions derived from ligandable drug target families, a percentage of processes or pathways found in an enrichment of gene data in a training model and in enriched lists of the plurality of predictions, at least one overlap between pathway enrichment or process enrichment data between the entities, a summary of relationships associated with the predictions to one or more objects in a database, at least one reduction to practice statement of association between the plurality of predictions and a disease context, and at least one connectivity associated with protein-protein interactions.

As another option, outputting the set of metrics for evaluation further comprising: displaying the set of metrics on an interface.

As another option, the outputted set of metrics are evaluated with at least one automated system configured to process or select one or more predictions based on at least one predetermined criterion associated with the outputted set of metrics.

As another option, said at least one automated system is associated with the predictive machine learning model.

As another option, evaluating the entities of the data source based on the outputted set of metrics.

As another option, wherein the plurality of predictions are generated in relation to said entities of a knowledge graph.

As another option, the plurality of predictions are generated using one or more pre-trained predictive machine learning models.

As another option, the set of metrics is adapted to be used with a predictive machine learning model.

As another option, the set of metrics are associated with a biomedical context or to be used to process data in a biomedical domain.

As another option, one or more metrics of the set of metrics are associated with evaluating an enrichment process or configured to determine whether the plurality of predictions is enriched.

As another option, said at least one display option are displayed in relation to the set of metrics in accordance with any of previous claims 14 to 19.

As another option, the interface device is configured to receive one or more inputs of entities associated with a knowledge graph.

As another option, in response to receiving said one or more inputs and following the output of the set of metrics, wherein an external application module configured to receive the outputted set of metrics and an associated metrics reference list from said at least one processor of the interface device.

As another option, a second application module is configured to receive the outputted set of metrics and the associated metrics reference list for a report publisher.

As another option, the report publisher is configured to collate and compile the received set of metrics and the associated metrics reference list to generate a representative report for visualising the set of metrics as display options on the interface device.

In the embodiments and aspects described above the server or computing device may comprise a single server/computing device or a network of servers/computing devices. In some examples the functionality of the server may be provided by a network of servers distributed across a geographical area, such as a worldwide distributed network of servers, and a user may be connected to an appropriate one of the network of servers based upon a user location.

The above description discusses embodiments and aspects of the invention with reference to a single user for clarity. It will be understood that in practice the system may be shared by a plurality of users, and possibly by a very large number of users simultaneously.

The embodiments and aspects described above are fully automatic. In some examples a user or operator of the system may manually instruct some steps of the method to be carried out.

In the described embodiments and aspects of the invention the system may be implemented as any form of a computing and/or electronic device. Such a device may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the method in hardware (rather than software or firmware). Platform software comprising an operating system or any other suitable platform software may be provided at the computing-based device to enable application software to be executed on the device.

Various functions described herein can be implemented in hardware, software, or any combination thereof. If implemented in software, the functions can be stored on or transmitted over as one or more instructions or code on a computer-readable medium. Computer-readable media may include, for example, computer-readable storage media. Computer-readable storage media may include volatile or non-volatile, removable or non-removable media implemented in any method or technology for storage of information such as computer readable instructions, data structures, program modules or other data. A computer-readable storage media can be any available storage media that may be accessed by a computer. By way of example, and not limitation, such computer-readable storage media may comprise RAM, ROM, EEPROM, flash memory or other memory devices, CD-ROM or other optical disc storage, magnetic disc storage or other magnetic storage devices, or any other medium that can be used to carry or store desired program code in the form of instructions or data structures and that can be accessed by a computer. Disc and disk, as used herein, include compact disc (CD), laser disc, optical disc, digital versatile disc (DVD), floppy disk, and blu-ray disc (BD). Further, a propagated signal is not included within the scope of computer-readable storage media. Computer-readable media also includes communication media including any medium that facilitates transfer of a computer program from one place to another. A connection, for instance, can be a communication medium. For example, if the software is transmitted from a website, server, or other remote source using a coaxial cable, fiber optic cable, twisted pair, DSL, or wireless technologies such as infrared, radio, and microwave are included in the definition of communication medium. Combinations of the above should also be included within the scope of computer-readable media.

Alternatively, or in addition, the functionality described herein can be performed, at least in part, by one or more hardware logic components. For example, and without limitation, hardware logic components that can be used may include Field-programmable Gate Arrays (FPGAs), Application-Program-specific Integrated Circuits (ASICs), Application-Program-specific Standard Products (ASSPs), System-on-a-chip systems (SOCs), Complex Programmable Logic Devices (CPLDs), etc.

Although illustrated as a single system, it is to be understood that the computing device may be a distributed system. Thus, for instance, several devices may be in communication by way of a network connection and may collectively perform tasks described as being performed by the computing device.

Although illustrated as a local device it will be appreciated that the computing device may be located remotely and accessed via a network or other communication link (for example using a communication interface).

The term ‘computer’ is used herein to refer to any device with processing capability such that it can execute instructions. Those skilled in the art will realise that such processing capabilities are incorporated into many different devices and therefore the term ‘computer’ includes PCs, servers, mobile telephones, personal digital assistants and many other devices.

Those skilled in the art will realise that storage devices utilised to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realise that by utilising conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages. Variants should be considered to be included into the scope of the invention.

Any reference to ‘an’ item refers to one or more of those items. The term ‘comprising’ is used herein to mean including the method steps or elements identified, but that such steps or elements do not comprise an exclusive list and a method or apparatus may contain additional steps or elements.

As used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices.

Further, as used herein, the term “exemplary” is intended to mean “serving as an illustration or example of something”.

Further, to the extent that the term “includes” is used in either the detailed description or the claims, such term is intended to be inclusive in a manner similar to the term “comprising” as “comprising” is interpreted when employed as a transitional word in a claim.

The figures illustrate exemplary methods. While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

Moreover, the acts described herein may comprise computer-executable instructions that can be implemented by one or more processors and/or stored on a computer-readable medium or media. The computer-executable instructions can include routines, sub-routines, programs, threads of execution, and/or the like. Still further, results of acts of the methods can be stored in a computer-readable medium, displayed on a display device, and/or the like.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of a preferred embodiment is given by way of example only and that various modifications may be made by those skilled in the art. What has been described above includes examples of one or more embodiments. It is, of course, not possible to describe every conceivable modification and alteration of the above devices or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

1. A computer-implemented method of generating a set of metrics for evaluating entities used with a predictive machine learning model, the computer-implemented method comprising:
selecting one or more sets of entities from a data source;
generating a plurality of predictions aggregated from said one or more sets of entities using one or more pre-trained predictive models;
selecting a subset of predictions from the plurality of predictions based on said one or more sets of entities in relation to the data source;
extracting metadata from the data source associated with the subset of predictions, wherein the metadata comprises entity metadata and predicted metadata;
generating the set of metrics based on the metadata extracted and the subset of predictions; and
outputting the set of metrics for evaluation.

2. The computer-implemented method of claim 1, wherein the subset of predictions comprises top predictions ranked in relation to said one or more pre-trained predictive models.

3. The computer-implemented method of claim 2, wherein the set of metrics are generated based on said top predictions and associated metadata.

4. The computer-implemented method of claim 3, wherein said associated metadata comprising said predicted metadata.

5. The computer-implemented method of claim 1, wherein said one or more pre-trained predictive models are adapted for a biomedical context.

6. The computer-implemented method of claim 5, wherein said one or more pre-trained predictive models are trained using biomedical data.

7. The computer-implemented method of claim 6, wherein said biomedical data is enriched or has undergone a process of enrichment using data further extracted from one or more sources.

8. The computer-implemented method of claim 1, further comprising:
selecting said one or more set of entities from the data source that comprises a knowledge graph; and extracting metadata from the knowledge graph, wherein the knowledge graph is configured to encode data related to a biomedical domain or a field corresponding to the biomedical domain.

9. The computer-implemented method of claim 1, wherein the set of metrics are based on one or a combination of: at least one overlap between the plurality of predictions, a set top correlations of objects in a database, a set of top processes, at least one correlation of the predictions with metadata associated with database objects, a proportion of the predictions derived from ligandable drug target families, a percentage of processes or pathways found in an enrichment of gene data in a training model and in enriched lists of the plurality of predictions, at least one overlap between pathway enrichment or process enrichment data between the entities, a summary of relationships associated with the predictions to one or more objects in a database, at least one reduction to practice statement of association between the plurality of predictions and a disease context, and at least one connectivity associated with protein-protein interactions.

10. The computer-implemented method of claim 1, wherein outputting the set of metrics for evaluation further comprising: displaying the set of metrics on an interface.

11. The computer-implemented method of claim 1, wherein the outputted set of metrics are evaluated with at least one automated system configured to process or select one or more predictions based on at least one predetermined criterion associated with the outputted set of metrics.

12. The computer-implemented method of claim 11, wherein said at least one automated system is associated with the predictive machine learning model.

13. The computer-implemented method of claim 1, further comprising: evaluating the entities of the data source based on the outputted set of metrics.

14. An interface device for displaying a set of metrics, the interface device comprising:
a memory;
at least one processor configured to access the memory and perform operations according to claim 1;
an output model configured to output the set of metrics; and
an interface configured to display at least one display option comprising:
an overlap option, a top pathways option, a model-literature option, a ligandability option, a mistake targets option, a pathway enrichment option, a process enrichment option, a disease pathway recall option, a disease process recall option, a disease benchmark interactions option, a reduction to practice presence option, and a protein-protein interaction connectivity option.

15. The interface device of claim 14, wherein said at least one display option are displayed in relation to the set of metrics, the set of metrics comprising:
at least one overlap between a plurality of predictions;
a set of top correlations of objects in a database;
a set of top processes;
at least one correlation of the predictions with metadata associated with database objects;
a proportion of the predictions derived from ligandable drug target families;
a percentage of processes or pathways found in an enrichment of gene data in a training model and in enriched lists of the plurality of predictions;
at least one overlap between pathway enrichment or process enrichment data between the entities,
a summary of relationships associated with the predictions to one or more objects in a database;
at least one reduction to practice statement of association between the plurality of predictions and a disease context; and
at least one connectivity associated with protein-protein interactions.

16. The interface device of claim 14, wherein the interface device is configured to receive one or more inputs of entities associated with a knowledge graph.

17. The interface device of claim 16, in response to receiving said one or more inputs and following the output of the set of metrics, wherein an external application module configured to receive the outputted set of metrics and an associated metrics reference list from said at least one processor of the interface device.

18. The interface device of claim 17, wherein a second application module is configured to receive the outputted set of metrics and the associated metrics reference list for a report publisher.

19. The interface device of claim 18, wherein the report publisher is configured to collate and compile the received set of metrics and the associated metrics reference list to generate a representative report for visualising the set of metrics as display options on the interface device.

20. A system for comparing and evaluating a plurality of predictions based on a set of metrics, the system comprising:
an input module configured to receive one or more sets of entities and associated metadata from a data source;
a processing module configured to predict, based said one or more sets of entities in relation to the data source, the plurality of predictions, wherein the plurality of predictions are ranked in a subset set of predictions;
a computation module configured to compute the set of metrics based on the plurality of prediction and the associated metadata, wherein the computation is performed using one or more pre-trained predictive models; and
an output module configured to present the set of metrics for evaluation.

21. The system of claim 20, wherein the set of metrics for evaluating the plurality of predictions comprises:
at least one overlap between a plurality of predictions;
a set of top correlations of objects in a database;
a set of top processes;
at least one correlation of the predictions with metadata associated with database objects;
a proportion of the predictions derived from ligandable drug target families;
a percentage of processes or pathways found in an enrichment of gene data in a training model and in enriched lists of the plurality of predictions;
at least one overlap between pathway enrichment or process enrichment data between the entities,
a summary of relationships associated with the predictions to one or more objects in a database;
at least one reduction to practice statement of association between the plurality of predictions and a disease context; and
at least one connectivity associated with protein-protein interactions.

22. The system of claim 20, wherein the system is configured to:
select the one or more sets of entities from the data source;
generate a plurality of predictions aggregated from said one or more sets of entities using one or more pre-trained predictive models;
selecting a subset of predictions from the plurality of predictions based on said one or more sets of entities in relation to the data source;
extract metadata from the data source associated with the subset of predictions, wherein the metadata comprises entity metadata and predicted metadata;
generate the set of metrics based on the metadata extracted and the subset of predictions; and
output the set of metrics for evaluation.
